(19) United States
(12) Patent Application Publication
(10) Pub. No.: US 2026/0013929 A1
(43) Pub. Date: Jan. 15, 2026

(54) DEVICES AND METHODS FOR FRACTIONAL TREATMENT OF TISSUE UTILIZING TISSUE PROPERTIES

(71) Applicant: POLLOGEN LTD., Tel Aviv (IL)

(72) Inventors: Amikam Gershonowitz, Tel Aviv (IL); Eyal Ben Esti, Tel Aviv (IL); Mordechai Moti Elnekave, Tel Aviv (IL); Zipora Ozer Armon, Tel Aviv (IL)

(21) Appl. No.: 19/332,815

(22) Filed: Sep. 18, 2025

Related U.S. Application Data
(63) Continuation of application No. PCT/IL2024/050767, filed on Aug. 1, 2024.
(60) Provisional application No. 63/530,561, filed on Aug. 3, 2023.

(51) Int. Cl. A61B 18/14; A61B 18/00; A61B 18/12

(52) U.S. Cl. CPC A61B 18/14; A61B 18/1206; A61B 2018/00077; A61B 2018/00083; A61B 2018/0016; A61B 2018/00184; A61B 2018/0047; A61B 2018/00577; A61B 2018/00589; A61B 2018/00702; A61B 2018/0072; A61B 2018/00732; A61B 2018/00761; A61B 2018/00875; A61B 2018/0091; A61B 2018/126; A61B 2018/1266; A61B 2018/1425

(57) ABSTRACT

A skin tissue treatment device and method comprising at least one first elongated electrode having a first polarity and configured for receiving electric current from an electric current source and for being inserted into the skin tissue to one or more depths. Also comprising at least one second electrode located in vicinity of the at least one first elongated electrode and configured to receive the electric current from the electric current source at a second and opposite polarity to the at least one first elongated electrode and an actuating mechanism being connected to at least to the at least one first elongated electrode and configured to spatially move the first elongated electrode along one or more axes. The at least one controller connected to the at least one first elongated electrode, the at least one second electrode, the electric current source, and the actuating mechanism; and the at least one controller being configured to: control the electric current source; actuate the actuating mechanism; and selectively apply fractional treatment of at least one of skin tissue ablation, skin tissue coagulation, or skin tissue mechanical insertion to the skin tissue.

### CROSS REFERENCE TO RELATED APPLICATIONS

This application is a continuation application of PCT Application No. PCT/IL2024/050767, filed Aug. 1, 2024, which claims the benefit of priority of U.S. Provisional Patent Application No. 63/530,561, filed Aug. 3, 2024, entitled “DEVICES AND METHODS FOR FRACTIONAL TREATMENT OF TISSUE UTILIZING TISSUE PROPERTIES”, the contents of which are incorporated herein by reference in its entirety.

### TECHNOLOGICAL FIELD

The present disclosure is in the medical, aesthetic, field and relates specifically to devices and methods for treatment of tissue, such as the skin, for aesthetic and/or cosmetic purposes. More specifically, the present disclosure relates to devices and methods for fractional treatment of skin tissue of a subject.

### BACKGROUND

Various skin tissue treatment techniques for skin tissue rejuvenation are available. Some treatments involve fractional skin tissue treatment, a term used to describe a form of treatment that creates a discrete array of relatively small treatment spots in the skin tissue and leaves sites of healthy and untreated skin tissue around the treatment spots. The treatment spots may be created by optical (e.g. laser), electromagnetic (e.g. radio frequency), sound (e.g. Ultrasound) or other energies/modalities. At each treatment spot a micro damage is created in the skin tissue. The micro damage in the skin tissue at the treatment spots triggers a natural healing response of the skin tissue. The intact healthy skin tissue surrounding the treatment spots provides basis for healing the micro damage.

Examples for fractional skin tissue treatment can be found in WO2021234609A1 and WO2021234605A1 assigned to the assignee of the present disclosure and are incorporated herein by reference in their entirety.

### SUMMARY OF INVENTION

The presently disclosed subject matter provides a device and methods for skin tissue treatment. The present disclosure provides a skin device comprising: an electric current source configured to provide Alternating Current (AC); Direct Current (DC) or any combination thereof; at least one first elongated electrode having a first polarity and configured for receiving electric current from the electric current source and for being inserted into the skin tissue to one or more depths; at least one second electrode located in vicinity of the at least one first elongated electrode and configured to receive the electric current from the electric current source at a second and opposite polarity to the at least one first elongated electrode; an actuating mechanism being connected to at least the at least one first elongated electrode and configured to spatially move the first elongated electrode along one or more axes; at least one controller connected to the at least one first elongated electrode, the at least one second electrode, the electric current source, and the actuating mechanism. The at least one controller being configured to: control the electric current source; actuate the actuating mechanism; and selectively apply treatment of skin tissue ablation, skin tissue coagulation, or skin tissue mechanical insertion to the skin tissue.

In one aspect of the disclosed subject matter, the device further comprises a handpiece configured to be held by a user. The handpiece comprising; the at least one first elongated electrode; the at least one second electrode; and the actuating mechanism. The device wherein the handpiece further comprises a disposable tip removably connectable to the handpiece with the disposable tip comprising at least one of: the at least first elongated electrode; or the at least one second electrode. Also, device further comprising a sensing system configured to transmit sensing data of the skin tissue to the controller, wherein the sensing data is indicative of at least one of electrical impedance or mechanical impedance.

In another aspect of the disclosed subject matter, there device has the at least one second electrode configured to be at least one of: elongated shaped configured to pass into the skin tissue; flat shaped configured to contact a surface of the skin tissue; or flat shaped having perforated holes configured to pass at least one first elongated electrode through to the skin tissue. An aspect of the device wherein a plurality of the at least one elongated electrode is configured in a comb-like one dimensional array, and wherein a plurality of the comb-like one dimensional arrays are arranged beside each other along a second axis, forming a two-dimensional array of the at least one elongated electrode. Another aspect of the device wherein the at least one elongated electrode is configured to have on the surface at least one of a plurality of insulated patterns on and further comprises a plurality of conductive points along the plurality insulated patterns, and wherein the plurality of insulated patters is configured on the at least second electrode in an elongated shape. In yet another aspect of the device wherein the actuator is additionally configured to rotate the at least one elongated electrode around a longitudinal axis of the at least one elongated electrode.

In one aspect of the disclosed subject matter, There is a skin tissue treatment method comprising: providing an electric current source configured to provide Alternating Current (AC); Direct Current (DC) or any combination thereof; providing at least one first elongated electrode having a first polarity and configured for receiving electric current from the electric current source and for being inserted into the skin tissue to one or more depths; providing at least one second electrode located in vicinity of the at least one first elongated electrode and configured to receive the electric current from the electric current source at a second and opposite polarity to the at least one first elongated electrode; providing an actuating mechanism being connected to at least the at least one first elongated electrode and configured to spatially move the first elongated electrode along one or more axes; providing connected to the at least one first elongated electrode, the at least one second electrode, the electric current source, and the actuating mechanism; providing at least one controller; placing the at least one elongated electrode and the at least one second electrode into contact with the skin tissue. The method further comprising selectively activating, by the at least one controller, the electric current source for applying an electric current profile to at least one of the at least one first elongated electrode or the at least one second electrode, the actuating mechanism to move the at least one elongated electrode for a predetermined distance into or out of the skin tissue. The method wherein treatment of skin tissue ablation, skin tissue coagulation, or skin tissue mechanical insertion is selectively applied to the skin tissue.

In another aspect the method further comprises: activating, by the at least one controller, the electric current source to provide an electric current profile to the at least one first elongated electrode and the at least one second electrode, for a predetermined first time period; deactivating, by the at least one controller, the electric current source; activating the actuating mechanism, by the at least one controller, to move the at least one elongated electrode for a predetermined distance into or out of the skin tissue; and activating, by the at least one controller, the actuating mechanism to move the at least one elongated electrode for a second distance.

In one aspect, the method further comprises: providing a sensing system configured to transmit sensing data of the skin tissue that is indicative of at least one of electrical impedance or mechanical impedance; and adjusting, based on the sensing data and by the at least one controller, at least one of; the electric current received by the at least one first elongated electrode, or the electric current received by the at least the at least one second electrode or activating the actuating mechanism.

In yet another aspect, the method, further comprises adjusting, based on the sensing data and by the at least one controller, the electric current received to selectively apply skin tissue ablation or skin tissue coagulation. Wherein the method further comprises providing a plurality of the at least one elongated electrode and are divided into two or more groups of elongated electrodes; and the method further comprises actuating, by the at least one controller, the two or more groups to move in different activation patterns into and out of the skin tissue. In some aspects, the plurality of the at least one elongated electrode may comprise at least one of: equal lengths; different lengths; equal transverse cross section areas or shapes; or different transverse cross section areas/shapes. Also, an aspect of the method wherein the actuating mechanism is configured to additionally rotate the at least one elongated electrode around the longitudinal axis of the at least one elongated electrode; and the method further comprises actuating, by the at least one controller, the actuating mechanism to move the at least one elongated electrode a predetermined distance into or out of the skin tissue while rotating around the longitudinal axis of the at least one elongated electrode.

A final aspect of the method is provided, wherein the at least one second electrode configured to be a flat shape having perforated holes that are configured to pass the at least one first elongated electrode through; and the method further comprising actuating, by the at least one controller, the actuating mechanism to move the at least one elongated electrode a predetermined distance through the perforated holes into the skin tissue.

### BRIEF DESCRIPTION OF THE ACCOMPANYING DRAWINGS

In order to better understand the subject matter that is disclosed herein and to exemplify how it may be carried out in practice, embodiments will now be described, by way of non-limiting example only, with reference to the accompanying drawings.

FIG. 1 illustrates a non-limiting exemplary embodiment of a device according to the presently disclosed subject matter.

FIG. 2 illustrates a non-limiting exemplary embodiment of a method according to the presently disclosed subject matter.

FIG. 3 illustrates non-limiting examples of fractional skin tissue treatments utilizing the devices and methods of the presently disclosed subject matter.

FIGS. 4A-4I illustrate various shapes for an elongated electrode and a second electrode used in the devices, according to non-limiting embodiments of the presently disclosed subject matter.

FIG. 5 is a close-up view of flat and elongated electrodes used in the devices, according to non-limiting embodiments of the presently disclosed subject matter.

FIG. 6 is a perspective view of a distal end portion of an elongated electrode used in the devices, according to non-limiting embodiments of the presently disclosed subject matter.

### DETAILED DESCRIPTION OF EMBODIMENTS OF THE INVENTION

In accordance with the present disclosure, treatment of tissue may involve one or more of the following: tissue ablation, where the tissue is ablated and ceases to exist, e.g. due to being subjected to high thermal energy; tissue coagulation, where the tissue is damaged to a degree that promotes renewal and rejuvenation, e.g. when being subjected to a thermal energy lower than that causing ablation; and mechanical injury of tissue. The mechanical injury typically employs mechanical insertion or mechanical removal of elongated electrodes, hereinafter mechanical insertion.

Typically, ablation alone may use elongated electrodes to both pierce the skin and move the electrodes deeper into the skin tissue. Ablation as a skin tissue piercing and movement mechanism usually requires short needles to ablate well. Also, typically mechanical insertion with pressure from sharp or pointed elongated electrodes may be used alone to pierce the skin and move them deeper into the skin tissue. In some embodiments of the current disclosure, piercing and movement into the skin tissue is done by ablation and/or mechanical insertion pressure and is employed in any combination of simultaneously, alternately or in any desired activation pattern thereof.

Properties of the skin tissue that can be utilized to optimize treatment include, for example, electrical impedance (resistance) and mechanical impedance. For example, properties of the skin tissue and tissue layers below, such as fat and muscle, can vary with respect to their electrical or/and mechanical impedance. Accordingly, the fractional treatment is optimized in the spatial, and possibly in the time, domain(s) to achieve an effective treatment outcome.

Fractional treatment creates a discrete array of relatively small treatment spots in the skin tissue and leaves sites of healthy and untreated skin tissue around the treatment spots. Typically, fractional treatment employs spaced apart multiple elongated electrodes inserted into skin tissue so that only small areas on the skin surface are treated. Fractional treatment, as referred to herein may be a one, two or three-dimensional fractional treatment. In some embodiments, the fractional treatment is along a single axis, e.g. a propagation axis of an elongated electrode, needle, or pin. In some embodiments, a plurality of elongated electrodes is provided, being arranged one beside another with respect to the treated skin tissue, to execute a fractional treatment across at least two axes of defined alignment, e.g. vertical, to the propagation axis of the needles or pins. In this disclosure, “electrode” (in the singular) or “electrodes” (in the plural) are understood to mean one or more electrodes.

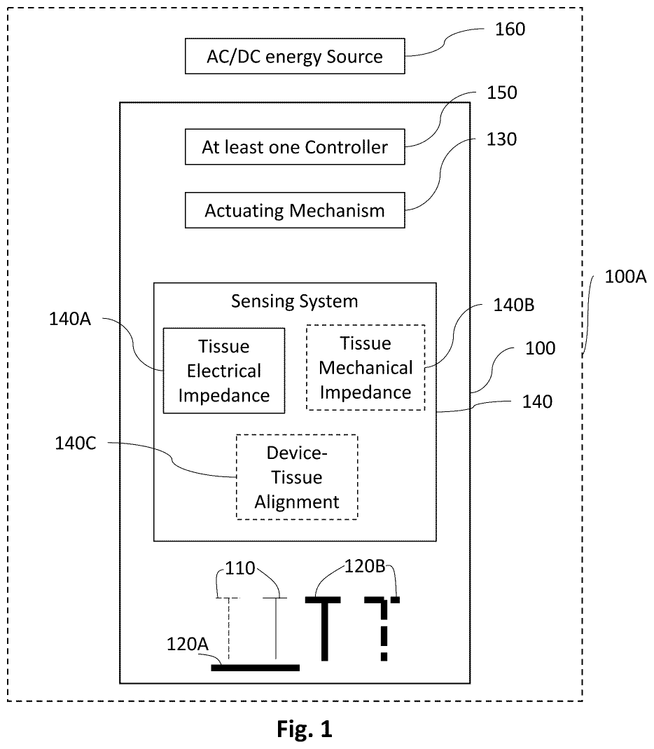

In the block diagram of FIG. 1, a non-limiting example of a device for skin tissue fractional treatment configured in accordance with the presently disclosed subject matter is illustrated. In some embodiments, device 100 is configured for fractional skin tissue treatment, the fractional treatment being tailored to the specific, localized, properties of the skin or treated tissue. In particular, Alternating Current (AC) (e.g., without being limited thereto, at Radio Frequency (RF) range) and/or Direct Current (DC) may be employed. Therefore, an AC and/or DC electric current source 160 may be part of the device as illustrated in 100A or may be outside and connected to the device 100.

In some embodiments, the electric current source is configured to controllably provide AC and DC simultaneously, alternately or in any desired activation pattern thereof. That is, the electric current source 160 may provide an electrical energy or current to produce thermal energy and the resulting damage of the treated skin tissue. In some embodiments, the device is at least partially configured as a handpiece device. For example, the device's elements above may be housed within a handpiece interfacing with the skin tissue site to be treated.

In some embodiments, the device 100 further comprises one or more electrodes or micro electrodes 110, 120A, and 120B configured to receive and deliver electrical current. In some embodiments, the electrical current is used, solely or in addition to mechanical fractional treatment of the tissue, as further described below. It is noted that, ablation of skin tissue may be done employing an AC signal producing thermal heat, while a DC current may ablate tissue based on a chemical reaction. The electrodes in device 100 may be configured with elongated shapes 110 and 120B. The elongated shapes, in some embodiments, enable electrode insertion into deep layers of the tissue and optionally below the skin tissue into fat and/or muscle layers. To this end, elongated electrodes may have a length dimension much larger than their width and depth dimensions and may be referred to as micro needles or pins, either having a solid or hollow transverse cross section.

In some embodiments, first elongated shape electrode or electrodes 110 are configured for receiving electric current at a first polarity and for being inserted into the skin tissue to one or more depths. In some embodiments, second electrodes 120A or 120B are configured for receiving electric current being opposite to the first polarity. In some embodiments, second electrodes 120A are flat electrodes. Flat second electrodes in combination with elongated electrodes are described in U.S. Pat. No. 11,717,679 filed 19 May 2021 and U.S. Pat. No. 11,963,710 filed 19 May 2021, both assigned to the assignee of the present disclosure and are incorporated herein by reference in their entirety.

In some embodiments, second electrodes 120B are also configured as elongated electrodes. In some embodiments, second electrodes are in the vicinity of the first elongated electrodes. In some embodiments, elongated electrodes 110 and second electrodes when elongated electrodes 120B comprise insulated sections, represented by the dashed line figures of 110 and 120B.

In some embodiments, the device 100 comprises a disposable tip (not shown) removably connectable to the handpiece and housing comprising at least one first elongated electrode of the first polarity. In some embodiments, the disposable tip also houses at least one second electrode of the second polarity. In some embodiments, the device is configured to supply or dispense into the skin tissue ingredient(s) or substance(s). In some embodiments, the elongated electrodes are configured with one or more hollow channels to deliver the ingredient(s) or substance(s) into the skin tissue. The hollow channels 21 may be in an elongated electrode 11 as illustrated in FIG. 6, where elongated electrode 11 represents any elongated electrode including 110 and 120B.

In some embodiments, the device further comprises an actuating mechanism 130 configured to spatially move first and/or second elongated electrodes along one or more axes. In some embodiments, a plurality of the elongated electrodes is arranged in a comb-like one dimensional array. In some embodiments, a plurality of the comb-like one dimensional arrays are arranged beside each other along a second axis, forming a two-dimensional array of the first and/or second elongated electrodes.

Typically, random distribution of the fractional treatment is advantageous for treating specific skin tissue conditions. In some embodiments, the actuating mechanism 130 is further configured to arrange the plurality of the elongated electrodes in any three-dimensional arrangement, for example to be distributed either randomly or according to a predetermined pattern in the x-y plane and progressed into the skin tissue along the z-direction. In some embodiments, an actuator is also configured to rotate elongated electrodes around the longitudinal axis of the elongated electrode. For example, the actuator may move the elongated electrode for a predetermined distance into or out of the skin tissue while rotating each individual elongated electrode around the longitudinal axis of the elongated electrode.

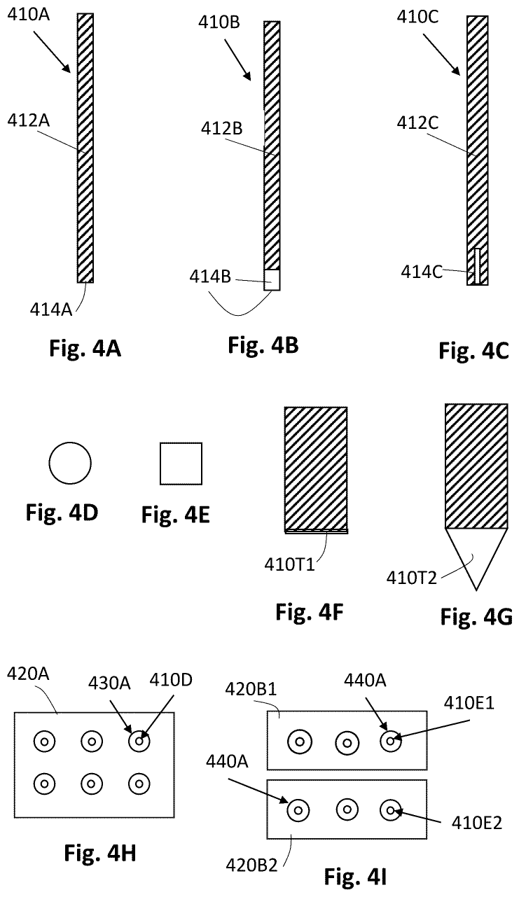

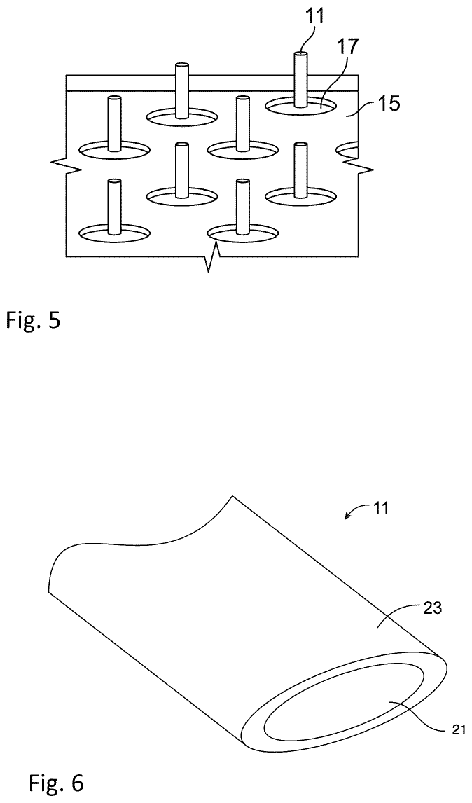

In some embodiments, the second electrodes are flat electrodes aligned at a certain degree, e.g. 90°, with respect to the longitudinal axis of the first elongated electrodes. By way of specific example, FIG. 5 illustrates the flat second electrode 15 with elongated first electrodes 11 at 90° to the flat second electrode. The flat second electrodes may be configured to contact the surface of the skin tissue site where the first elongated electrodes are inserted. In some embodiments, the flat second electrodes are configured to be independently activated against the first elongated electrodes. In some embodiments, the flat second electrodes are perforated with holes 430A in FIG. 4H, 440A in FIGS. 41 and 17 in FIG. 5, through which one or more of the first elongated electrodes is/are passed on the way into the skin tissue site located beneath the flat second electrodes.

In some embodiments, the second electrodes are elongated electrodes 120B configured as the first elongated electrodes and operable for insertion and withdrawal from the skin tissue, such that the electric current is applied inside the skin tissue between the first elongated electrodes and the second electrodes. It is appreciated that in such case, the second electrodes, being elongated electrodes, forms part of the elongated electrodes arrangement, in other words all the features described herein with respect to the first elongated electrodes are applicable also for the second electrodes. In this case, the second elongated electrodes may be activated in any order of opposite polarities to apply an electric current treatment to the skin tissue.

In some embodiments, the elongated electrodes of first and/or second electrodes have equal lengths or different lengths. In some embodiments, the elongated electrodes of first and/or second electrodes have equal transverse cross section areas/shapes or have different transverse cross section areas/shapes. In some embodiments, the elongated electrodes of the first and/or second are divided into two or more groups having different lengths or transverse cross section areas/shapes. In some embodiments, different groups of elongated electrodes are actuated by different activation patterns.

In some embodiments, the device further comprises a sensing system or mechanism 140 configured to provide sensing data indicative of at least one of skin tissue electrical impedance, skin tissue mechanical impedance, or any combination thereof. In some embodiments, the sensing mechanism 140 further comprises a device-skin tissue alignment system 140C; electrical impedance sensing system 140A; and a skin tissue mechanical impedance sensing system 140B. In some embodiments, at least part of the sensing system 140 is mounted on/embedded in at least one elongated electrode and/or at least one second electrode (not shown), at the active portion (i.e., the location of the activated electric current) of the electrode. In some embodiments, and in the case of electrical impedance, the active portion of the electrode may form at least part of the sensing system.

The device 100, in some embodiments, comprises a controller 150. In this disclosure, “controller” (in the singular) is understood to mean one or more controllers or processors that may be hosted on a single computer or whether the features and functions of the controller are distributed over a plurality of networked computers. In some embodiments, the controller 150 further comprises associated user interfaces including but not limited to a display or an input which may include a keyboard and/or mouse (not shown). In some embodiments, the controller is configured to receive sensing data from the sensing mechanism 140; control the electric current source 160; and the actuating mechanism 130 to selectively apply at least one treatment of skin tissue ablation, skin tissue coagulation and skin tissue mechanical insertion to the treated skin tissue. In some embodiments, the controller is configured to apply skin tissue treatment including skin tissue ablation, skin tissue coagulation, skin tissue mechanical insertion, either separately or in any combination thereof.

In some embodiments, the controller controls at least one of amplitude, and frequency in case of RF signal of the electric current source. In some embodiments, the controller controls and adjusts at least one of RF amplitude and frequency of the electric current source either manually or automatically based on real-time sensing data received from the sensing system. It is known that, regardless of the tissue type, the electrical impedance increases with the depth and the distance between the at least one elongated electrode and the at least one second electrode. In some embodiments, the controller controls and adjusts the at least one of amplitude and frequency of the electric current source either manually or automatically based on stored data or look-up table(s) relating to kind of the tissue as a function of depth (e.g. the depth of the elongated electrode).

In some embodiments, the controller is configured to control the electric current source and the actuating mechanism to cause movement of at least one elongated electrode inside the skin tissue as a function of skin tissue ablation or skin tissue mechanical insertion/piercing. In some embodiments, the skin tissue ablation or skin tissue mechanical insertion/piercing is based on sensing data, e.g. impedance measurements, received from the sensing system. In some embodiments, the at least one controller is configured to turn the electric current source on and off alternately while the at least one of the first and/or second elongated electrode is moved by the actuating mechanism inside the skin tissue, according to predetermined one or more programs, thereby resulting in treatment of the skin tissue along the propagation axis of the at least one elongated electrode.

In some embodiments, electrical impedance sensing system and the controller are configured to determine the profile (amplitude and/or frequency) of the electric current to achieve the desired localized skin tissue ablation or coagulation effect. For example, the controller may adjust the amplitude and/or frequency to achieve a predetermined lateral and/or three-dimensional localized skin tissue ablation or coagulation. For example, increasing the frequency of an RF signal, at a predetermined amplitude selected to cause skin tissue coagulation, will result in a coagulation zone closer to the at least one first and/or second elongated electrode and vice versa.

In some embodiments, a mechanical impedance measurement system and the controller is configured to determine the actuating mechanism parameters, such as the moving force amplitude and/or frequency and/or rate of insertion to skin tissue, to cause a desired localized movement profile of the first and/or second elongated electrode inside the skin tissue. In some embodiments, the mechanical impedance is determined based on the electrical current consumption of the actuating mechanism.

In some embodiments, the sensing system provides at least one of electrical impedance or mechanical impedance sensing data indicative of coupling or alignment between the device and the skin tissue to be treated, for example as a safety measure. The controller may be configured to automatically activate a treatment session, once and only if a coupling/alignment condition is met, by activating the actuating mechanism to deploy at least one elongated electrode into the skin tissue and activating the electric current source to apply an ablation or coagulation treatment to the skin tissue. For example, the sensing system may measure a mechanical impedance measurement by incorporating one or more of the following non-limiting examples: pressure sensors, velocity sensors, acoustic sensors, optical sensors. The sensing system may, additionally or alternatively, measure an electrical impedance measurement indictive of the coupling/alignment, such as by dedicated electrode sensors located at the interface between the device and the skin tissue (not shown). In some embodiments, the coupling/alignment condition is that an entire surface of the device that interfaces with the skin tissue is coupled/aligned, without air gaps therebetween. In some embodiments, the controller is configured to automatically turn off the electric current source and pull the first and second elongated electrodes out of the skin tissue once the coupling/alignment condition ceases to exist.

In some embodiments, the controller is configured to activate the electric current source and the actuating mechanism in a periodic pattern. The actuating mechanism may be activated to deploy elongated electrodes into the skin tissue and then retract elongated electrodes out of the skin tissue. Simultaneously, the electric current source may be activated to apply a treatment, according to a predetermined pattern, e.g. continuously or intermittently, when the elongated electrodes are within the skin tissue. Sensing data received from the sensing system, such as impedance data, may be used by the controller to determine the parameters of the actuating mechanism, such as the deployment and retraction velocities (which can be constant or variable), and to determine the activation parameters of the electric current source, such as the amplitude, frequency, and timing patterns of the treatment electrical current. In some embodiments, the periodic pattern is such that it is repeated with time gaps in between each activation cycle to allow the user of the device to move the device between different skin tissue sites, in a so-called “stamping” method. In some embodiments, controlling the electrical current parameters, e.g. by multi-pulse regime, enables avoiding bulk heating, undesirable over-treating, and/or burning/damaging the surrounding skin tissue.

In the flow diagram of FIG. 2, a non-limiting example of a method for skin tissue fractional treatment configured in accordance with the presently disclosed subject matter is illustrated.

Method 10a may include providing at least one first elongated electrode having a first polarity.

Method 10b may include providing at least one second electrode located in vicinity of the at least one first elongated electrode and having a second polarity opposite the first elongated electrode polarity.

Method 10c may include providing an electric current source connected to the at least one first elongated electrode and the at least one second electrode.

Method 10d may include providing an actuating mechanism being connected at least to the at least one first elongated electrode and configured to spatially move the at least one first elongated electrode along one or more axes.

Method 10e may include contacting the skin tissue with the at least one first elongated electrode and the at least one second electrode.

Method 10f may include sequentially applying, by at least one controller, the following two steps in a predetermined number of times:
Step f1 may include activating, by at least one controller, the electric current source to provide an electric current profile to the at least one first elongated electrode and the at least one second electrode, for a predetermined first time period; and
Step f2 may include deactivating, by the at least one controller, the electric current source, and activating the actuating mechanism to move at least the at least one first elongated electrode for a predetermined distance into or out of the skin tissue.

Method 10g may include activating, by the at least one controller, the actuating mechanism to move at least the at least one elongated electrode for a second distance. In some embodiments. Method 10g may be done during step (f1).

Method 10h may include adjusting, by the at least one controller, the electric current profile based on the sensing data. Method 10g may be done during step (f1).

In some embodiments, the method above may include activating a RF electrical source of the electrical current source to ablate the skin tissue, and after the elongated electrodes are inside the skin tissue activate the DC electrical source.

In some embodiments, and when rotation of the elongated electrodes is employed, an activation pattern may result in a directional thermal damage of the skin tissue in both x-y directions (due to rotation) and z direction (due to advancement into or out of the skin tissue). In some embodiments, the thermal damage can have a helix shape, i.e. a three-dimensional spiral curve. In some embodiments, the elongated electrodes include a plurality of conductive points along a plurality insulated surface, as described below. Thus, enabling more complex profiles of treatment as desired, such as double or triple helix thermal damage zone(s).

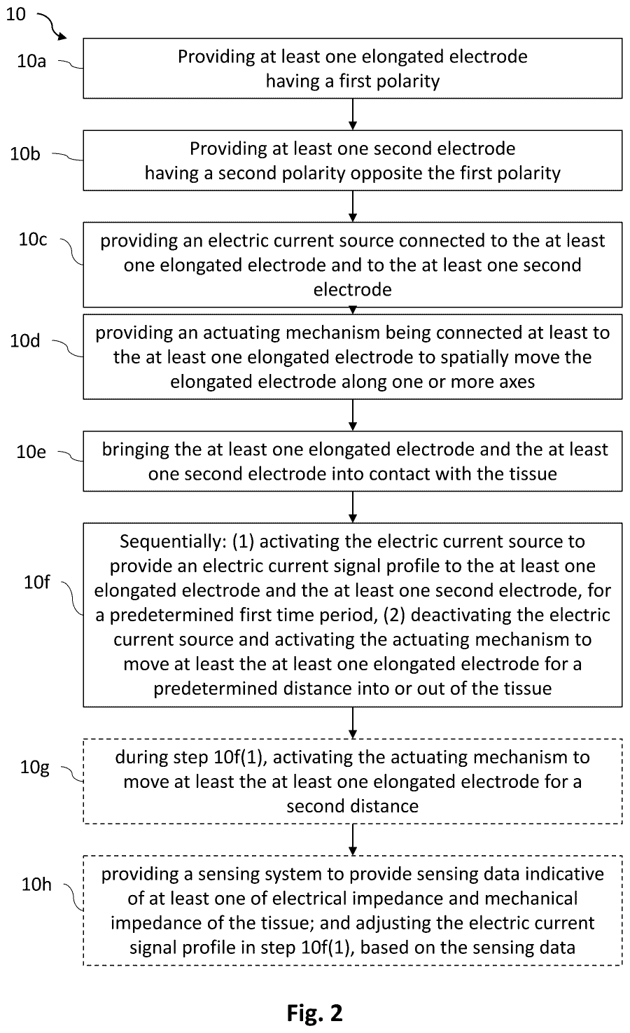

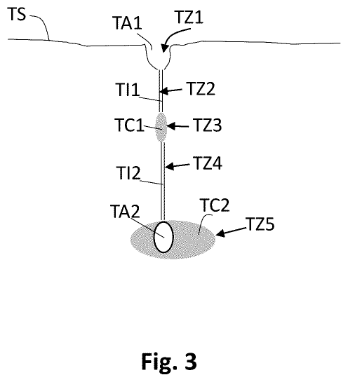

In FIG. 3, different examples of tissue effects of the fractional treatment of skin tissue in accordance with different aspects of the presently described subject matter are illustrated. For simplicity, the diagram shows different tissue effects of fractional treatment by one elongated electrode with an opposite polarity working against a second flat electrode positioned on skin tissue surface TS (both elongated and flat electrodes are not shown). The fractional treatment effects are along propagation axis of the elongated electrode inside, and alongside the skin tissue. The exemplified skin tissue effects may be obtained, as another aspect of the invention, in view of momentary skin tissue impedance measurements taken at the tip of the elongated electrode.

At skin tissue zone TZ1, skin tissue ablation TA1 is obtained by a suitable electrical energy. At skin tissue zone TZ2, skin tissue mechanical insertion TI1 is obtained by piercing the skin tissue with mechanical movement of the elongated electrode into the skin tissue. At skin tissue zone TZ3, skin tissue coagulation TC1 is obtained by a suitable electrical energy. At skin tissue zone TZ4, skin tissue mechanical insertion TI2, with a longer length than TZ2, is obtained by piercing the skin tissue with mechanical progression of the elongated electrode. At skin tissue zone TZ5, both skin tissue ablation TA2 and directional skin tissue coagulation TC2 are obtained by suitable electrical energies. According to one aspect of the invention, the coagulation energy may be applied before the ablation energy and according to another aspect of the invention, the ablation energy may be applied before the coagulation energy. According to the present invention, different combinations of mechanical piercing lengths, ablative electrodes insertion and coagulated tissue zones may be applied.

In FIGS. 4A-4G, non-limiting examples of first and/or second elongated electrodes configured in accordance with the presently disclosed subject matter are illustrated.

FIGS. 4A-4C are side views illustrating the insulation of the external surface of the at elongated electrodes. In some embodiments, elongated electrodes are insulated, fully or partially, along a proximal external surface thereof.

FIG. 4A shows an insulation profile 412A that extends for the whole length L of the elongated electrode 410A. The conductive, uninsulated part 414A is at the bottom side of the elongated electrode. In this embodiment, the electric current is exiting the elongated electrode only at the bottom side in the direction of the propagation axis of the elongated electrode. It is noted that the electric current flows between the bottom side 414A of the elongated electrode and the at least one second electrode. When the latter is a flat electrode placed on surface of the skin tissue site, the electric current flows between a small area of the tip of the elongated electrode, thus having a high current density, and a large area of the flat second electrode, thus having a low current density. This configuration results in a higher and more controllable and precise tissue effect in the vicinity of the tip of the elongated electrode and a lower or insignificant tissue effect adjacent the flat second electrode. When the latter is another elongated electrode inserted into the skin and adjacent to the first elongated electrode and having an opposite electrical polarity during RF application, a treatment effect, either by ablation and/or by coagulation, on the tissue adjacent the tips of the paired elongated electrodes may be achieved.

FIG. 4B shows an insulation profile 412B that extends for most part of the length of the elongated needle electrode 410B. The conductive part 414B extends along part of the length and bottom side of the needle electrode. In some embodiments, the elongated electrode is insulated along whole external surface except for one or more elongated strips extending proximally from the bottom end of the elongated electrode. Typically, this configuration results in a directional flow of the electric current into/out of the elongated electrode.

FIG. 4C shows an elongated electrode being insulated all over its external surface apart from a longitudinal strip 414C kept uninsulated and thus conductive. In some embodiments, the actuating mechanism is further configured to selectively rotate the elongated electrode around its longitudinal axis to select the direction of the directional flow of the electric current from the strip 414C to its paired second electrode, having an opposite electrical polarity during RF operation. Such a second electrode may be a flat electrode on the skin surface and/or one or more adjacent elongated electrodes. In some embodiments, based on the electrical impedance measurements, the at least one controller determines the directional flow of the electric current to achieve a desired treatment effect.

In some embodiments, the elongated electrode comprises an insulated external elongated member and at least one internal conductive elongated member controllably projectable from the insulated external elongated member (not shown). In some embodiments, the internal conductive elongated members are resilient yet still capable of penetrating the skin tissue either by ablation and/or mechanically. This configuration enables controlling the effective conductive portion of the elongated electrode by distally projecting a desired length of the internal conductive elongated member out of the insulated external elongated member.

In some embodiments, the insulated external elongated member includes a grooved portion in the form of an elongated strip extending at the distal side of the insulated external elongated member, as illustrated in FIG. 4C, 414C. The insulated external elongated member may be rotatable by the actuating mechanism with respect to the internal conductive elongated member, thereby enabling a desired side of the internal conductive elongated member, through the elongated strip, to control the directional flow and amplitude/density of the electric current.

In some embodiments, first and/or second elongated electrodes have a flat side body, e.g. the cross-section shape is rectangular or polygonal as illustrated in FIG. 4E. FIG. 4E more particularly illustrates a rectangular transverse cross-section of the electrode.

In some embodiments, the first and/or second elongated electrodes have a curved side body, such as round (circular or oval) transverse cross-section of the electrode as illustrated in FIG. 4D.

In some embodiments, the elongated first and/or second electrodes have a blunt distal, and as illustrated in FIG. 4F with a flat tip 410T1. The blunt distal end may be flat or curved.

In some embodiments, the elongated electrodes have a sharp or a pointed distal end configured for piercing skin tissue, such as FIG. 4G with the pointed tip 410T2.

FIG. 4H is a bottom view of a single flat second electrode 420A, as described above, and an array of, for example, six first elongated electrodes 410D passing through holes 430A formed in the single flat second electrode. The six elongated electrodes are electrically isolated from the flat second electrode. Any number of the first elongated electrodes may be activated against the single flat second electrode at any point in time. In another example, at least some of the six elongated electrodes may be activated with opposite polarities, i.e. part of them functions as a second elongated electrode.

FIG. 4I illustrates two flat second electrodes 420B1 and 420B2, each having three holes 440A through which two arrays 410E1 and 410E2 of three first elongated electrodes are passed. In addition to the possibilities of FIG. 4H, this enables also activating the two flat second electrodes against each other and against the array of elongated electrodes passing through the other flat second electrode.

Accordingly, the presently disclosed subject matter enables tailored fractional treatment of skin tissue, including any combination of ablation, coagulation, and mechanical insertion, based on localized skin tissue impedance (electrical and/or mechanical) measurements.

As an aid to understanding, the detail description may contain usage of the introductory phrases “at least one” and “one or more”. However, the use of such phrases should not be construed to imply that the introduction by the indefinite articles “a” or “an” limits any particular embodiment containing such to disclosure containing only one such recitation, even when the same includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce descriptions. In addition, even if a specific number of an introduced description is explicitly recited, those skilled in the art will recognize that such recitation should typically be interpreted to mean at least the recited number (e.g., the bare recitation of “two recitations,” without other modifiers, typically means at least two recitations, or two or more recitations.)

1. A skin tissue treatment device comprising:
at least one first elongated electrode having a first polarity and configured for being inserted into the skin tissue to one or more depths;
at least one second electrode having an opposite polarity to the at least one first elongated electrode and configured to be positioned on a surface of the skin tissue, wherein the at least one second electrode comprises at least one perforation to allow passage of the at least one first elongated electrode through the at least one second electrode to the skin tissue;
an electric current source configured to provide electric current to the at least one first elongated electrode and the at least one second electrode;
an actuating mechanism being connected to at least one first elongated electrode and configured to spatially move the at least one first elongated electrode along one or more axes; and
at least one controller operatively connected to the at least one first elongated electrode, the at least one second electrode, the electric current source, and the actuating mechanism;
wherein the at least one controller is configured to cause an insertion of the at least one first elongated electrode into the skin tissue, wherein said insertion comprises activating, by the at least one controller:
skin tissue ablation using a suitable electrical current provided by the electric current source via the at least one first elongated electrode; and
mechanical insertion of the at least one first elongated electrode into the skin tissue by piercing the skin tissue with mechanical movement of the at least one first elongated electrode into the skin tissue caused by the actuating mechanism.

2. The device of claim 1, wherein the at least one second electrode being disposed orthogonally with respect to the at least one first elongated electrode.

3. The device of claim 1, wherein said activating skin tissue ablation and said mechanically inserting are performed individually or in combination, either simultaneously or sequentially.

4. The device of claim 1, wherein the at least one controller is further configured to control the electric current source to adjust an electric current profile at different depths of the at least one first elongated electrode within the skin tissue, to generate different treatment effects in different zones within the skin tissue.

5. The device of claim 4, wherein the electric current profile comprises one or more of an amplitude, a frequency, and a timing pattern of the provided electric current.

6. The device of claim 1, wherein the at least one controller is configured to selectively apply treatment of skin tissue ablation, skin tissue coagulation, and skin tissue mechanical insertion to the skin tissue.

7. The device of claim 6, wherein the selectively applied treatment is obtained by:
controlling the electric current source to adjust an amplitude of provided electric current to one first elongated electrode and the at least one second electrode, or the time of providing the electric current, to provide skin tissue ablative insertion or different levels of skin tissue coagulation;
controlling the actuating mechanism to control a distance of a mechanical movement of at least one first elongated electrode into or out of the skin tissue;
controlling the electric current source to adjust an amplitude of provided electric current to one first elongated electrode and the at least one second
whereby enabling applying different combinations of mechanical insertion lengths, skin tissue ablative insertion and coagulated tissue zones along a treatment area.

8. The device of claim 1, further comprising a sensing system configured to measure and transmit sensing data of the skin tissue to the at least one controller, wherein the sensing data is indicative of at least one of electrical impedance or mechanical impedance of the skin tissue.

9. The device of claim 8, wherein the at least one controller is configured to selectively activate different types of fractional treatment of the skin tissue having different tissue effects by the at least one first elongated electrode, in accordance with momentary sensing data surrounding a tip of the at least one first elongated electrode.

10. The device of claim 1, further comprising a handpiece configured to be held by a user, the handpiece comprising;
the at least one first elongated electrode;
the at least one second electrode; and
the actuating mechanism.

11. The device of claim 10, wherein the handpiece further comprises a disposable tip removably connectable to the handpiece with the disposable tip comprising at least one of:
the at least first elongated electrode; or
the at least one second electrode.

12. The device of claim 1, wherein a plurality of the at least one elongated electrode is configured in a comb-like one dimensional array.

13. The device of claim 12, wherein a plurality of the comb-like one dimensional arrays are arranged beside each other along a second axis, forming a two-dimensional array of the at least one elongated electrode.

14. The device of claim 1, wherein the at least one first elongated electrode comprises a series of conductive regions interleaved with insulated regions to enable different treatment profiles along the skin tissue surrounding the at least one first elongated electrode.

15. The device of claim 1, wherein the actuating mechanism is further configured to rotate the at least one elongated electrode around a longitudinal axis thereof.

16. The device of claim 1, wherein the electric current provided by the electric current source comprises an Alternating Current (AC), a Direct Current (DC) or any combination thereof; wherein the skin tissue ablation is done by a thermal reaction produced by an AC signal and/or a chemical reaction produced by a DC signal.

17. A skin tissue cosmetic treatment method comprising:
providing at least one first elongated electrode having a first polarity and configured for being inserted into the skin tissue to one or more depths;
providing at least one second electrode having an opposite polarity to the at least one first elongated electrode and configured to be positioned on a surface of the skin tissue, wherein the at least one second electrode comprises at least one perforation to allow passage of the at least one first elongated electrode through the at least one second electrode to the skin tissue;
providing an electric current source configured to provide electric current to the at least one first elongated electrode and the at least one second electrode;
providing an actuating mechanism being connected to at least one first elongated electrode and configured to spatially move the at least one first elongated electrode along one or more axes; and
providing at least one controller operatively connected to the at least one first elongated electrode, the at least one second electrode, the electric current source, and the actuating mechanism;
inserting, by the at least one controller, at least one first elongated electrode into the skin tissue, wherein said inserting comprises selectively activating, by the at least one controller:
skin tissue ablation using a suitable electrical current provided by the electric current source via the at least one first elongated electrode; and
mechanical insertion of the at least one first elongated electrode by piercing the skin tissue with mechanical movement of the at least one first elongated electrode into the skin tissue caused by the actuating mechanism.

18. The skin tissue cosmetic treatment method of claim 17, wherein the at least one second electrode being disposed orthogonally with respect to the at least one first elongated electrode.

19. The skin tissue cosmetic treatment method of claim 17, wherein said skin tissue ablation and said mechanical insertion are performed individually or in combination, either simultaneously or sequentially.

20. The skin tissue cosmetic treatment method of claim 17, further comprises:
controlling, by the at least one controller, the electric current source to adjust an electric current profile at different depths of the at least one first elongated electrode within the skin tissue, to generate different treatment effects in different zones within the skin tissue;
wherein the electric current profile comprises one or more of an amplitude, a frequency, and a timing pattern of the provided electric current.

21. The skin tissue cosmetic treatment method of claim 17, further comprising:
selectively activating, by the at least one controller,
the electric current source for applying an electric current profile to at least one of the at least one first elongated electrode or the at least one second electrode, and
the actuating mechanism to move the at least one elongated electrode for a predetermined distance into or out of the skin tissue,
whereby selectively applying cosmetic treatment of skin tissue ablation, skin tissue coagulation, and skin tissue mechanical insertion to the skin tissue.

22. The skin tissue cosmetic treatment method of claim 21, wherein said selectively applying the cosmetic treatment comprises:
controlling the electric current source to adjust an amplitude of provided electric current to one first elongated electrode and the at least one second electrode, or the time of providing the electric current, to provide skin tissue ablative insertion or different levels of skin tissue coagulation;
controlling the actuating mechanism to control a distance of a mechanical movement of at least one first elongated electrode into or out of the skin tissue;
controlling the electric current source to adjust an amplitude of provided electric current to one first elongated electrode and the at least one second
whereby enabling applying different combinations of mechanical insertion lengths, skin tissue ablative insertion and coagulated tissue zones along a treatment area.

23. The skin tissue cosmetic treatment method of claim 21, wherein said selectively applying the cosmetic treatment further comprises:
activating skin tissue ablation at a first skin zone, wherein the skin tissue ablation is obtained by a first electrical energy profile;
activating skin tissue mechanical insertion at a second skin zone, wherein the skin tissue mechanical insertion is obtained by piercing the skin tissue with mechanical movement of the elongated electrode into the skin tissue along the second skin zone; and
activating skin tissue coagulation at a third skin zone, wherein the skin tissue ablation is obtained by a second electrical energy profile;
wherein the first and second energy profiles are different.

24. The skin tissue cosmetic treatment method of claim 23, wherein said selectively applying the cosmetic treatment further comprises:
activating skin tissue mechanical insertion at a fourth skin zone, wherein the skin tissue mechanical insertion is obtained by piercing the skin tissue with mechanical movement of the elongated electrode into the skin tissue along the fourth skin zone, wherein a length of the mechanical insertion at the fourth skin zone is longer than a length of the mechanical insertion at the second skin zone.

25. The skin tissue cosmetic treatment method of claim 23, wherein said selectively applying the cosmetic treatment further comprises:
activating skin tissue ablation and skin tissue coagulation at a fourth skin zone, wherein the skin tissue ablation and skin tissue coagulation are obtained by a third energy profile.

26. The skin tissue cosmetic treatment method of claim 17, further comprising:
measuring, by a sensing system, sensing data of the skin tissue, wherein the sensing data is indicative of at least one of electrical impedance or mechanical impedance of the skin tissue; and
transmitting the sensing data to the at least one controller,
wherein the at least one controller is configured to selectively activate different types of fractional cosmetic treatment of the skin tissue having different tissue effects by the at least one first elongated electrode, in accordance with momentary sensing data surrounding a tip of the at least one first elongated electrode.

27. The skin tissue cosmetic treatment method of claim 17, wherein a plurality of the at least one elongated electrode is configured in a comb-like one dimensional array, wherein a plurality of the comb-like one dimensional arrays are arranged beside each other along a second axis, forming a two-dimensional array of the at least one elongated electrode.

28. The skin tissue cosmetic treatment method of claim 17, wherein the at least one first elongated electrode comprises a series of conductive regions interleaved with insulated regions to enable different treatment profiles along the skin tissue surrounding the at least one first elongated electrode.

29. The skin tissue cosmetic treatment method of claim 18, wherein the actuating mechanism is further configured to rotate the at least one elongated electrode around a longitudinal axis thereof.

30. The skin tissue cosmetic treatment method of claim 18, wherein the electric current provided by the electric current source comprises an Alternating Current (AC), a Direct Current (DC) or any combination thereof; wherein the skin tissue ablation is done by a thermal reaction produced by an AC signal and/or a chemical reaction produced by a DC signal.
